(19) United States
(12) Patent Application Publication
(10) Pub. No.: US 2024/0299207 A1
(43) Pub. Date: Sep. 12, 2024

(54) DIGITALLY PRINTED ARCHES WITH ELASTIC ADVANCEMENTS FOR ANTI-SNORING

(71) Applicant: G-Force, Inc, Phoenix, AZ (US)

(72) Inventor: David Silas Gergen, Phoenix, AZ (US)

(21) Appl. No.: 18/600,320

(22) Filed: Mar. 8, 2024

Related U.S. Application Data
(60) Provisional application No. 63/450,772, filed on Mar. 8, 2023.

(51) Int. Cl. A61F 5/56

(52) U.S. Cl. CPC A61F 5/566

(57) ABSTRACT

A digitally-printed anti-snoring dental device is provided. The device includes a unitary upper appliance comprising an upper tray, an upper right specialized attachment and an upper left specialized attachment; a unitary lower appliance comprising a lower tray, a lower right specialized attachment and a lower left specialized attachment; at least one elastic attached to the upper right specialized attachment and to the lower right specialized attachment; and at least one elastic attached to the upper left specialized attachment and to the lower right specialized attachment. Each of the upper right specialized attachment, the upper left specialized attachment, the lower right specialized attachment, and the lower left specialized attachment includes an open ball hook configured to retain the at least one elastic thereon.

### CROSS-REFERENCE TO RELATED APPLICATION

This Application claims the benefit of U.S. Provisional Application No. 63/450,772, filed Mar. 8, 2023. The disclosure of which is hereby incorporated by reference in its entirety.

### BACKGROUND

### 1. Field

Example embodiments relate to oral mandibular repositioners, and, more specifically, to digitally printed anti-snoring arches.

### 2. Description of Related Art

An estimated 45 percent of adults snore occasionally, while 25 percent snore regularly, causing serious disturbance to their own sleep as well as their bed partners. This condition occurs when the lower jaw and tongue fall back during sleep to partially block the airway. When the airway is partially blocked, respiration causes an increase in air speed through the airway. The increased air speed at the mouth and throat causes soft tissue of the uvula and soft palate to vibrate. This vibration causes the sound that is known as snoring.

A known treatment for snoring is use of an oral appliance which repositions the lower jaw to prevent this partial blocking of the airway. This general category of devices is known as oral mandibular repositioners.

Sleep apnea results from a partial-to-complete blockage of the airway. Increased air speed through the airway will cause an increase in dynamic pressure and a corresponding drop in static pressure. The decreased static pressure tends to further draw back the lower jaw and tongue to further block the airway. This blockage can increase to the point of becoming complete, which at least temporarily interrupts breathing. Thus, sleep apnea is also known as obstructive sleep apnea. Typically, the blockage occurs in the soft palate or the base of the tongue. Those suffering from this condition can awaken with coughing and gasping as they try to restore normal breathing.

One treatment for sleep apnea is the use of an oral appliance. The lower jaw should be allowed to move during sleep to prevent general discomfort and specifically to avoid soreness in the temporomandibular joints. Custom fabricated appliances might employ swivels or hinges to join the upper and lower palates while allowing the lower jaw to move. Fitting custom appliances is costly because they require significant expertise by a trained dentist or dental laboratory.

Most often the devices are bulky and uncomfortable to wear leading to patient noncompliance of the device. What is required with most dental devices is for the patient to see a dentist and capture a bite with an opening of approximately 5 mm and the mandible advanced to the incisal edge of the upper anterior teeth.

There are also various health problems caused by snoring including, but not limited to: hypertension, high blood pressure, stroke, excessive daytime sleepiness, emotional irritability, learning difficulties, mouth breathing, growth problems, restless sleep, weight gain, drowsy driving, excessive bruxism, difficulty waking up refreshed in the morning, poor appetite, adult-onset diabetes, bed partner disruption, acid reflux, dry mouth, and bad breath.

### SUMMARY

Example embodiments may address at least the above problems and/or disadvantages and other disadvantages not described above. Also, example embodiments are not required to overcome the disadvantages described above, and may not overcome any of the problems described above.

An aspect of an example embodiment provides a digitally-printed anti-snoring dental device is provided. The device includes a unitary upper appliance comprising an upper tray, an upper right specialized attachment and an upper left specialized attachment; a unitary lower appliance comprising a lower tray, a lower right specialized attachment and a lower left specialized attachment; at least one elastic attached to the upper right specialized attachment and to the lower right specialized attachment; and at least one elastic attached to the upper left specialized attachment and to the lower right specialized attachment. Each of the upper right specialized attachment, the upper left specialized attachment, the lower right specialized attachment, and the lower left specialized attachment includes an open ball hook configured to retain the at least one elastic thereon.

### BRIEF DESCRIPTION OF THE DRAWINGS

The above and/or other aspects will become apparent and more readily appreciated from the following description of example embodiments, taken in conjunction with the accompanying drawings, in which:

FIG. 1 illustrates an upper digitally-printed arch with specialized attachments according an example embodiment;

FIG. 2 illustrates a lower digitally-printed arch with specialized attachments according to an example embodiment;

FIG. 3 illustrates an upper and lower alliance together, according to an example embodiment;

FIG. 4 illustrates a specialized attachment an example embodiment;

FIG. 5 illustrates a lower left specialized attachment, a lower left posterior bite plane, a lower right posterior bite plane, and a posterior bite plane left, according to an example embodiment;

FIG. 6 illustrates a side view of a specialized attachment according to an example embodiment;

FIG. 7 illustrates a model during fabrication according to an example embodiment;

FIG. 8 illustrates an occlusal view of a specialized attachment according to an example embodiment;

FIG. 9 illustrates an upper model display and a lower model display according to an example embodiment;

FIG. 10 illustrates an occlusal view of a digital model according to an example embodiment;

FIG. 11 illustrates an occlusal view of a patient's jaw;

FIG. 12 illustrates a view of a patient's jaw with bite planes added;

FIG. 13 illustrates an occlusal view with blackouts intact and bite planes added;

FIG. 14 illustrates a setup of a posterior bite plane on a lower arch;

FIG. 15 illustrates a side view of a patient's jaw;

FIG. 16 illustrates an appliance with posterior bite plane in place according to an example embodiment; and

FIG. 17 illustrates a specialized attachment placed on an appliance according to an example embodiment.

### DETAILED DESCRIPTION

Reference will now be made in detail to example embodiments which are illustrated in the accompanying drawings, wherein like reference numerals refer to like elements throughout. In this regard, the example embodiments may have different forms and may not be construed as being limited to the descriptions set forth herein.

It will be understood that the terms “include,” “including”, “comprise, and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “and/or” includes any and all combinations of one or more of the associated listed items. Expressions such as “at least one of,” when preceding a list of elements, modify the entire list of elements and do not modify the individual elements of the list.

Various terms are used to refer to particular system components. Different companies may refer to a component by different names—this document does not intend to distinguish between components that differ in name but not function.

Matters of these example embodiments that are obvious to those of ordinary skill in the technical field to which these example embodiments pertain may not be described here in detail.

One or more example embodiments may provide a dental device for treating snoring in patients with mild to moderate snoring issues which impacts their health.

One or more example embodiments may provide a comfortable appliance providing a higher level of compliance among users.

An appliance according to one or more example embodiments do not require a dental bite registration.

The mouth/body seeks its own cover zone.

An appliance according to one or more example embodiments does have buccal attachments, but the protrusion is slight, keeping the device streamlined and comfortable to wear with little to no irritation

An appliance according to one or more example embodiments uses dental elastics with different degrees of strength, allowing the mandible to advance and find a comfortable position.

One or more example embodiments may also provide a 3D designed and printed dental sleep device that can be easily replaced for a patient without having to revisit a dental office for new impressions. This is achieved by using a digital copy of the original patient's scan of their dental sleep device, which is stored in the cloud on secure corporate servers. Therefore, if a patient loses or breaks their appliance, a new one can easily be reprinted and shipped to the patient based on their original scan.

Many snorers or nocturnal bruisers have bites that are over closed which causes overcompensation of the airway. The bite planes according to one or more example embodiments may assist in preventing this issue. An appliance according to one or more example embodiments may use the bite plane for more than just retrognathia profiles. An appliance according to one or more example embodiments may provide bite plates on both the upper and lower to open the vertical and to prevent over closure.

A bite plate according to one or more example embodiments may cover the posterior teeth on the bicuspids back to the first molar on the lower arch, to allow an appliance to glide forward more easily to advance the mandible when the elastics are engaged on the upper and lower.

One or more example embodiments may provide a dentist and patient another treatment option, of a snoring device that does not require adjustments by the dentist or patient.

A device according to one or more example embodiments is an anti-snoring device which is a three-dimensionally (3D) designed and printed dental sleep device comprising maxillary and mandibular clear trays (also referred to as appliances). The trays are 3D printed with an FDA approved splint material and including the newly designed specialized attachments to make one solid appliance.

A device according to one or more example embodiments allows synovial fluid to flow into the jaw joint. This lateral movement into the congular housing is how the synovial fluid works. It is a lubricant for the joint. Without lateral movement, the joint would have temporomandibular joint (TMJ) issues. Appliances in the past restricted this lateral movement and have caused joint problems. A device according to one or more example embodiments uses elastics to gently pull the mandible forward and the upper bite plane and lower bite plane make contact, which allows the patient's airway to remain open for steady airflow.

A posterior bite plane is added to the mandibular arch and maxillary to open the vertical slightly on patients that have class 2, deep class 2, retrognathic and class 3 occlusion, where class 1 refers to an ideal bite, class 2 refers to an overbite, and class 3 refers to a goldfish bite or a bulldog bite.

A device according to one or more example embodiments is configured to gently glide the mandible into a position, opens the vertical, and allows for airflow which prevents snoring. A patient is glided into a position which allows vertical and lateral movement where synovial fluid prevents TMJ or temporomandibular disorder (TMD) issues from occurring. A device according to one or more example embodiments may thus benefit both the consumer and professional markets.

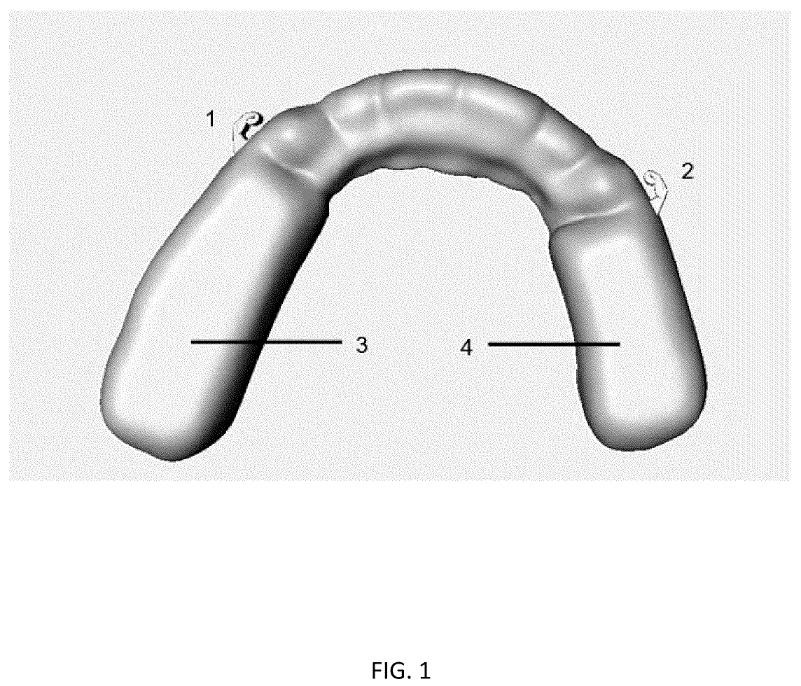

FIG. 1 illustrates an upper digitally-printed arch with specialized attachments according an example embodiment. The arch includes an upper right specialized attachment 1, an upper left specialized attachment 2, an upper right posterior bite plane 3, and an upper left posterior bite plane 4. The specialized attachments 1 and 2 are positioned between the cuspid and first bicuspid after the appliance is designed in computer-aided design (CAD) and/or computer-aided manufacturing (CAM) software. The upper right posterior bite plane 3, and an upper left posterior bite plane 4, from the first bicuspid to the first molar, show a height of approximately 1.5 mm. A plane is also created to attach the specialized attachments between the first bicuspid using a method in a CAD/CAM software.

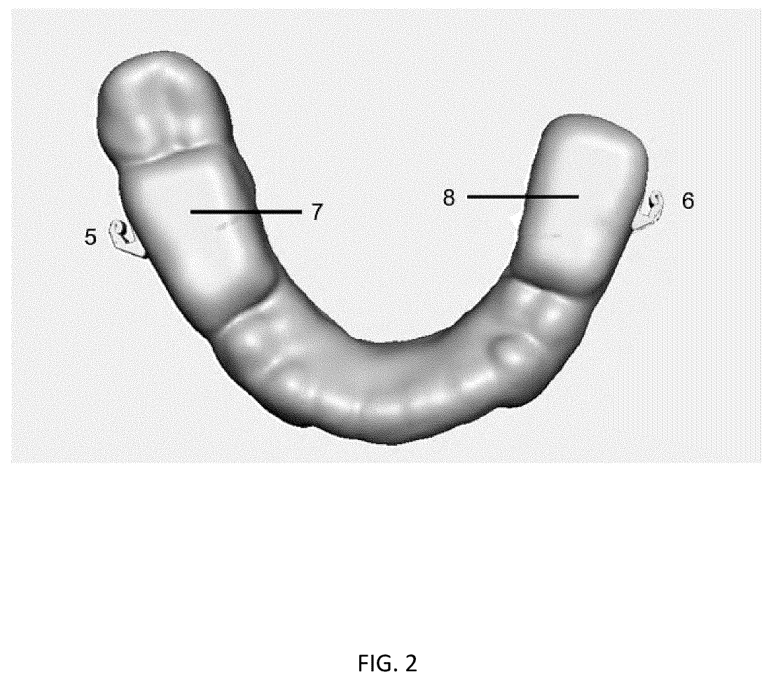

FIG. 2 illustrates a lower digitally-printed arch with specialized attachments according to an example embodiment. The arch includes a lower right specialized attachment 5, a lower left specialized attachment 6, a posterior right bite plane 7, and a posterior left bite plane 8. The a posterior right bite plane 7, and a posterior left bite plane 8, from the second bicuspid to the distal of the first molar, show a height of approximately 1.5 mm. A plane is also created to attach the specialized attachments at the mesial portion of the molar using a method in a CAD/CAM software.

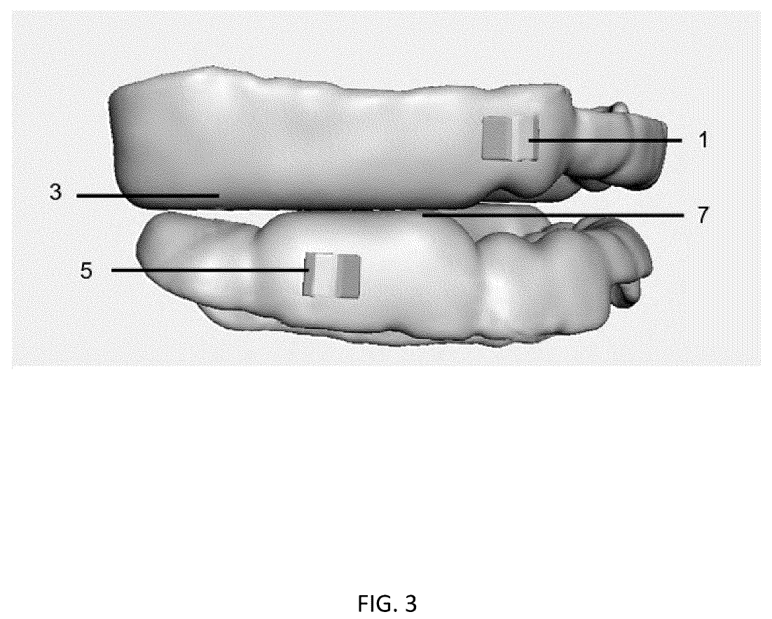

FIG. 3 illustrates an upper and lower alliance together, according to an example embodiment. This illustration shows the upper and lower appliances of FIGS. 1 and 2, respectively, without elastics, illustrating the upper right specialized attachment 1, the upper left specialized attachment 2, the lower right specialized attachment 5, and the lower left specialized attachment 6. Both the upper and lower appliances rest on the bite planes created in the software with the specialized attachments attached to the appliances.

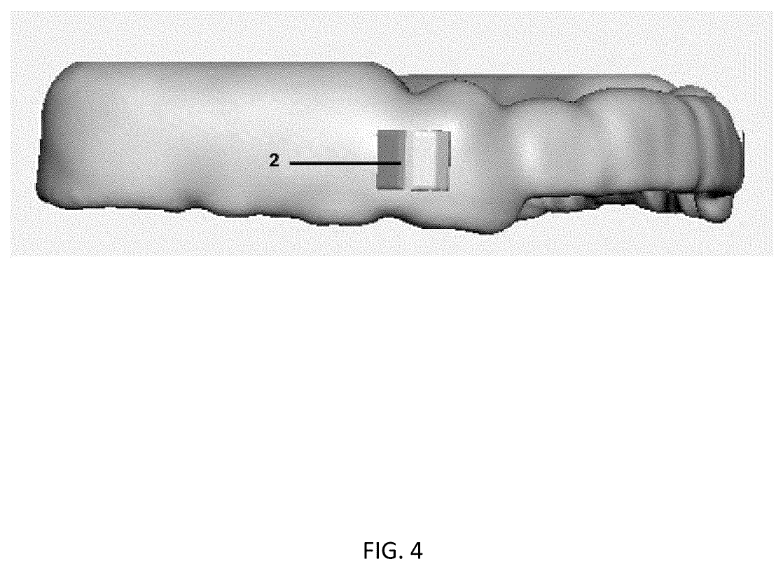

FIG. 4 illustrates an upside-down view of the upper left specialized attachment 2.

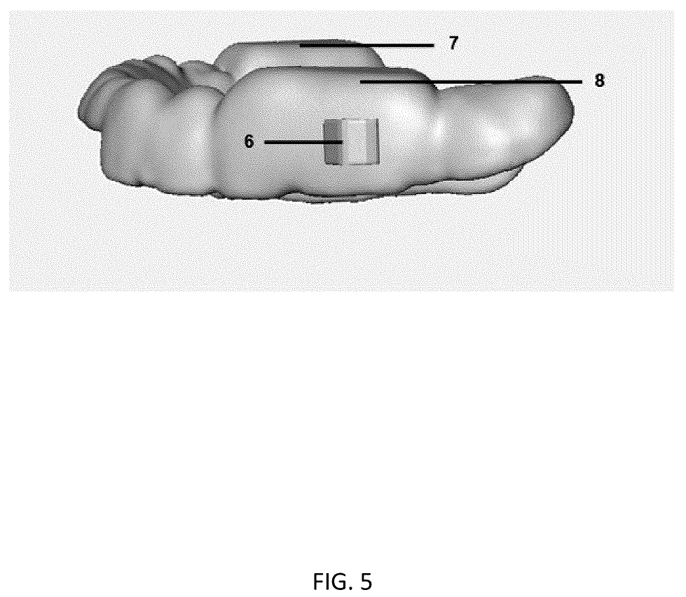

FIG. 5 illustrates a lower left specialized attachment 6, a lower left posterior bite plane (not labeled), a lower right posterior bite plane 7, and a posterior left bite plane 8.

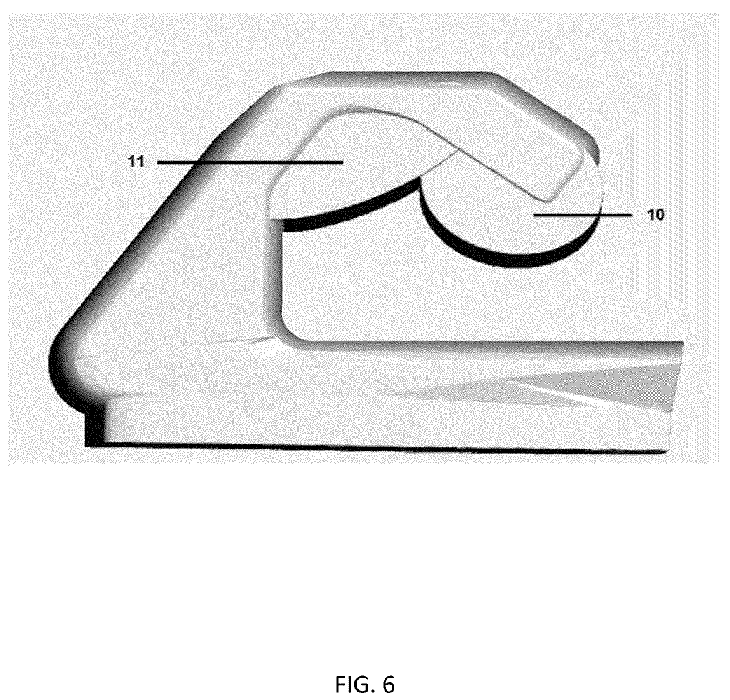

FIG. 6 illustrates a side view of a specialized attachment according to an example embodiment. This is an illustration of an example specialized attachment as used on an appliance according to an example embodiment. The attachment includes an open ball hook 10 which uses an anterior ball to aid in retention of an elastic. As positioned on the maxilla upper arch, the ball hook faces toward the mesial (anterior position), and as positioned on the mandible lower arch, the ball hook faces toward the distal (posterior position). An open space 11 is provided. When elastics are added, the upper and lower appliances are kept in place by the tension created by the elastics and the connection of the appliances to the teeth. Each of the specialized attachments is designed to house the elastic in such a way that the elastic will snap in and not disengage from the attachment. They are designed to have a low profile and not irritate the inside of the cheek when the patient is wearing the appliance. The specialized attachment is digitally added to the appliance file before 3D printing.

FIG. 6 shows the specialized attachment the for an appliance according to an example embodiment. The CAD/CAM drawing shows the front body hook 10 which allows an elastic to snap into place and rest in the inside diameter.

FIG. 3 shows the maxillary and mandibular appliances with the specialized attachments on both the lower and upper appliances and the bite planes resting against each other, opening the bite of the patient's jaw approximately 1.5 mm to 2.0 mm. The specialized attachment 5 is shown in a location in the mesial third region of the first molar. The specialized attachment 1 is shown in the interproximal of the cuspid area.

FIG. 1 shows an upper occlusal view with the specialized attachment in the interproximal of the cuspid and bicuspid on both sides of the jaw. The specialized attachment 5 is positioned approximately 1 mm away from the outer surface of the device. The elastic will be pulled through the opening of the specialized attachment to secure it in place and then attached to the lower appliance.

FIG. 2 shows an occlusal view of the lower appliance with the specialized attachments attached to the mesial third of the first molar area of the device. The front 5 of the specialized attachment 2 is placed approximately 1 mm away from the appliance surface to allow the elastic to pull through the opening, securing it in place. The elastic is placed on either the upper attachment or the lower attachment first and then stretched and attached to the opposing arch. Connecting the right side and the left side of the appliance.

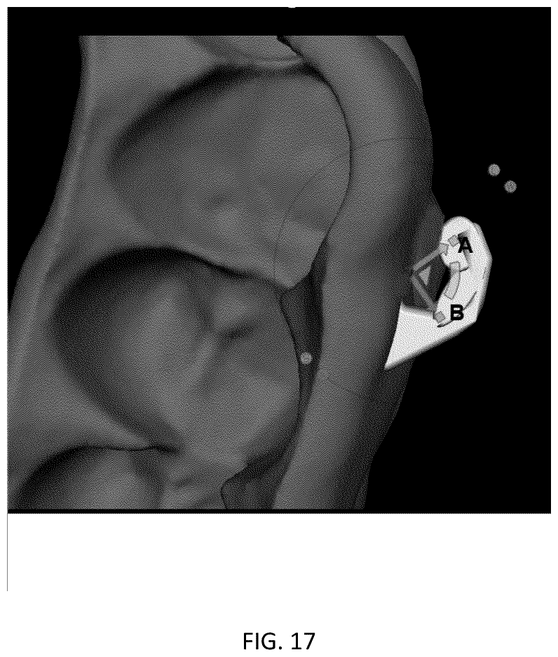

FIG. 17 shows the specialized attachment being integrated with the appliance. A distance of approximately 1.0 mm is shown from the front body hook to the splint body. Once the specialized attachment is placed the two mesh appliances are combined into one computer file.

The elastics that attach to the specialized attachment come with any of various forces to bring the necessary forces to pull the mandible forward. The elastics range from light (2.5 oz), Medium (3.5 oz) to heavy (4.5 oz). A patient can start with light and if snoring is still present can increase the force until snoring is reduced.

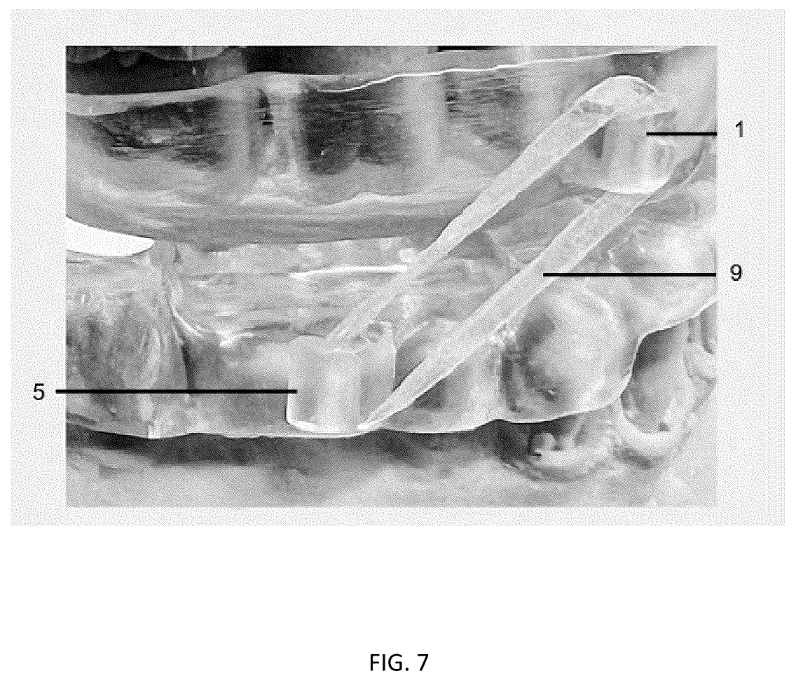

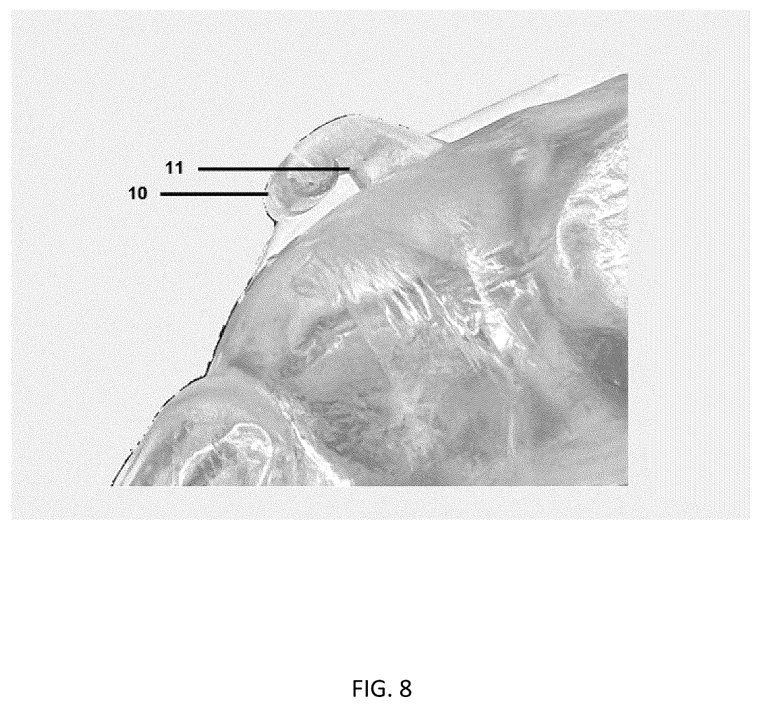

FIG. 7 illustrates a model during fabrication according to an example embodiment, including the upper right specialized attachment 1, the lower right specialized attachment 5, and an elastic band 9 attached to the upper right specialized attachment 1 and the lower right specialized attachment 5, the specialized attachments 1 and 5, as shown on the upper jaw, are placed between the cuspid and first bicuspid. The body hook part of the attachment is placed approximately 1 mm away from the surface of the appliance. This allows the elastic to lock in place and provide a larger inside diameter (FIG. 8, element 11) and for the elastic to rest inside without being impinged. Once the elastic(s) are placed this will advance the mandible gently in a forward position to aid in minimizing minimize snoring and sleep apnea.

FIG. 8 illustrates an occlusal view of a specialized attachment according to an example embodiment, including the ball hook 10 and the open space 11. This illustration shows the specialized attachment on the device after having been 3D printed.

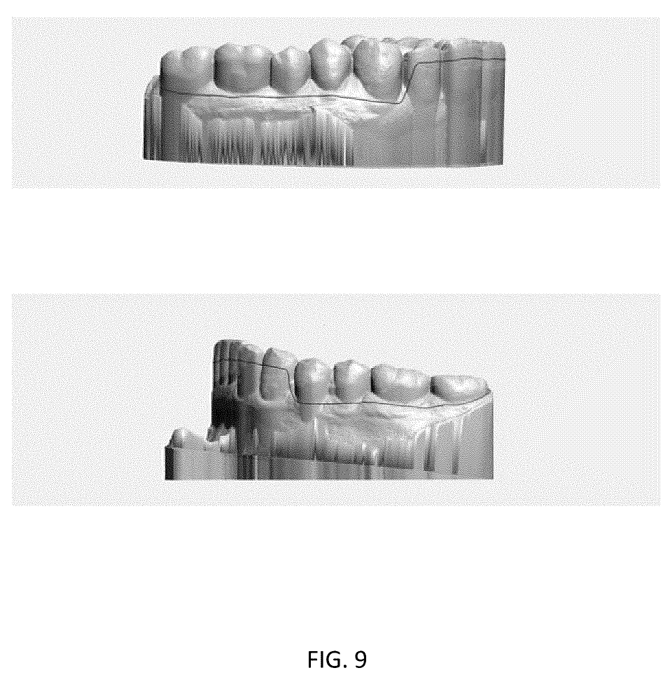

FIG. 9 illustrates an upper model display and a lower model display according to an example embodiment. Lines in the illustration illustrate where to block out the undercut for fabrication of the appliances. This figure illustrate a method of preparing the device for printing.

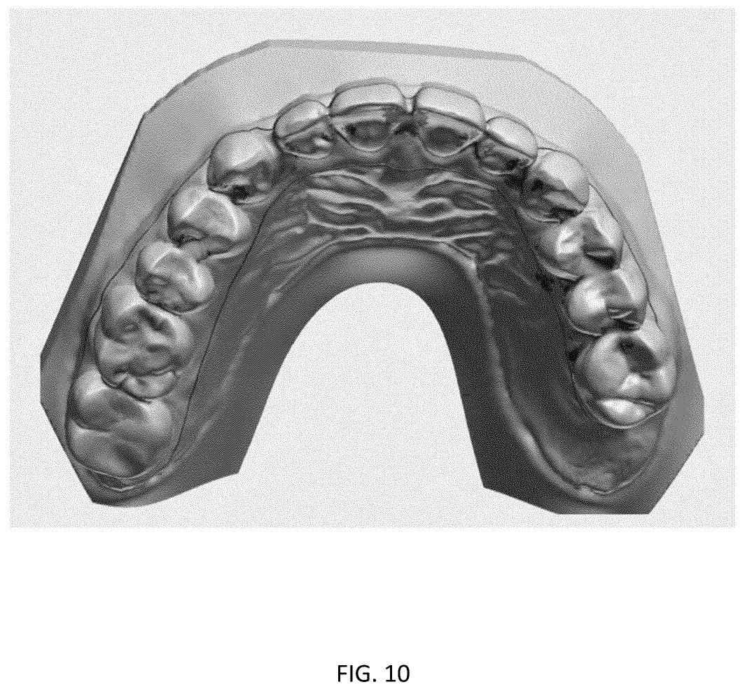

FIG. 10 illustrates an occlusal view of a digital model according to an example embodiment. In this illustration, lines are traced for blocking out undercuts and to provide a computer outline of a tray, start to end.

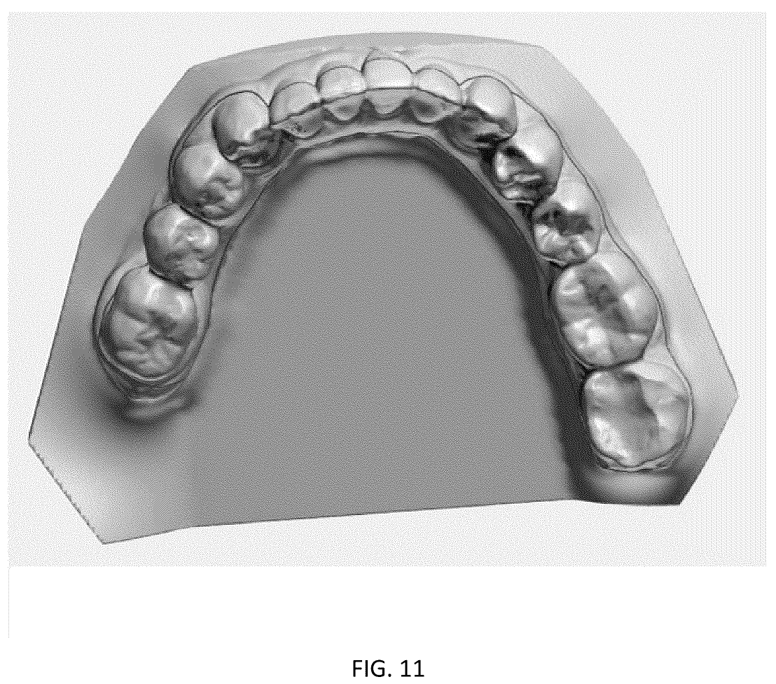

FIG. 11 illustrates an occlusal view of a patient's jaw in which locations for blocking out undercuts for fabrication are identified. This illustration shows an outline following the gingival margin until it connects to the line on the buccal portion. The bite plane will be raised approximately 1.5 mm using the round plane. This will allow a lab technician to raise both sides of the device evenly.

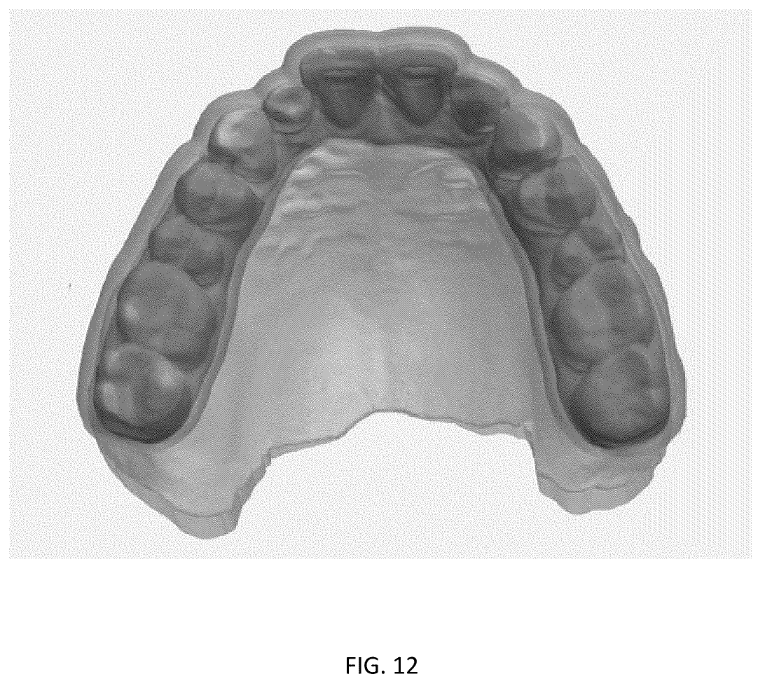

FIG. 12 illustrates a view of a patient's jaw with bite planes added. The maxillary jaw is drawn from starting at the second molar (if present) to the gingival of the teeth and starting at the mesial of the cuspid, it will be drawn to the incisal third of the facial lateral to lateral and continued around to the lingual area of the patient's teeth.

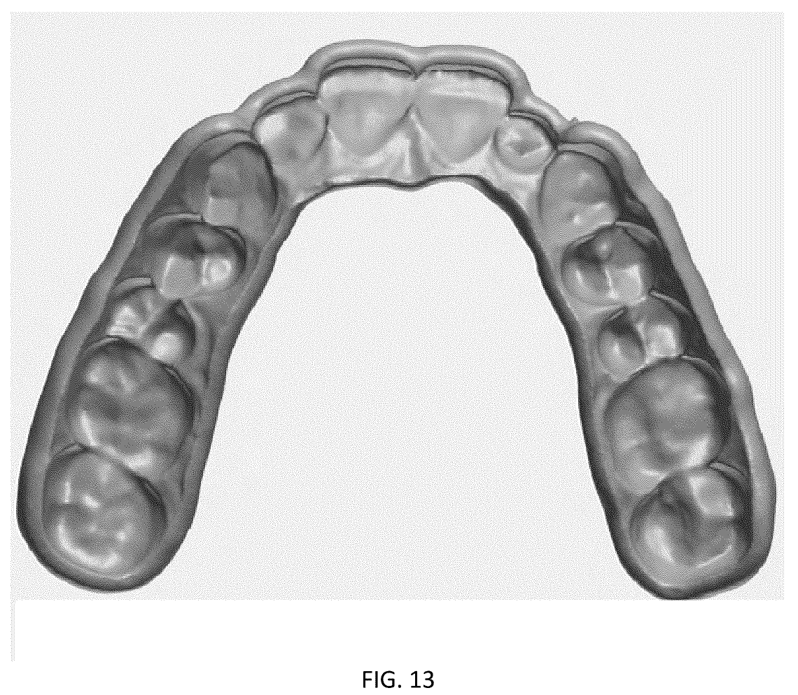

FIG. 13 illustrates an occlusal view with blackouts intact and bite planes added.

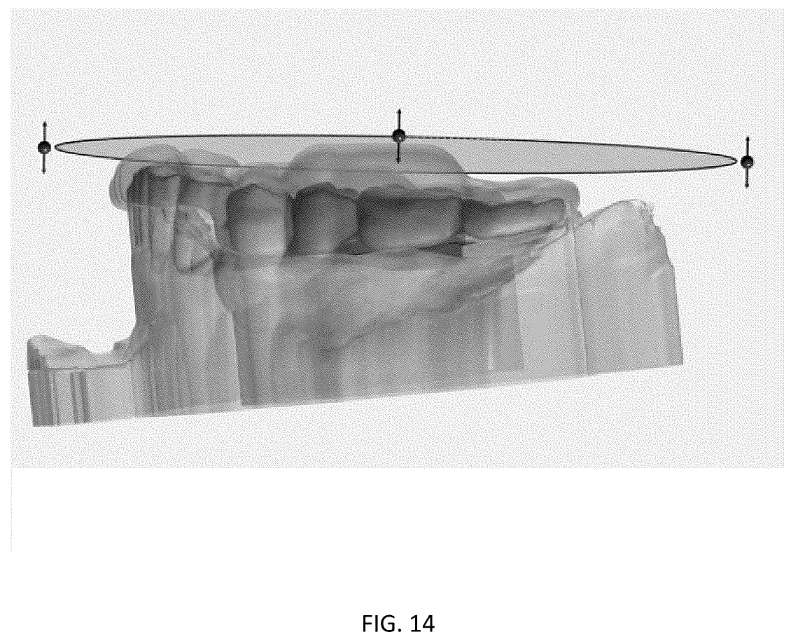

FIG. 14 illustrates a setup of a posterior bite plane on a lower arch, as used for fabrication of an appliance according to an example embodiment.

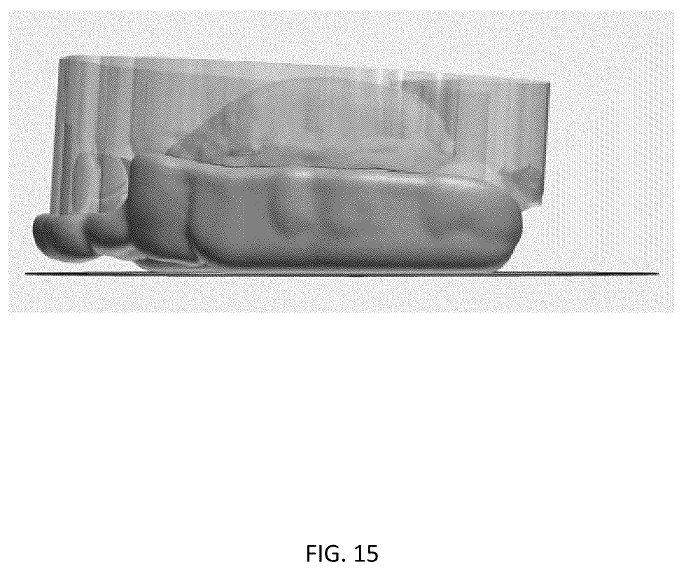

FIG. 15 illustrates a side view of a patient's jaw showing the flatness of the posterior table.

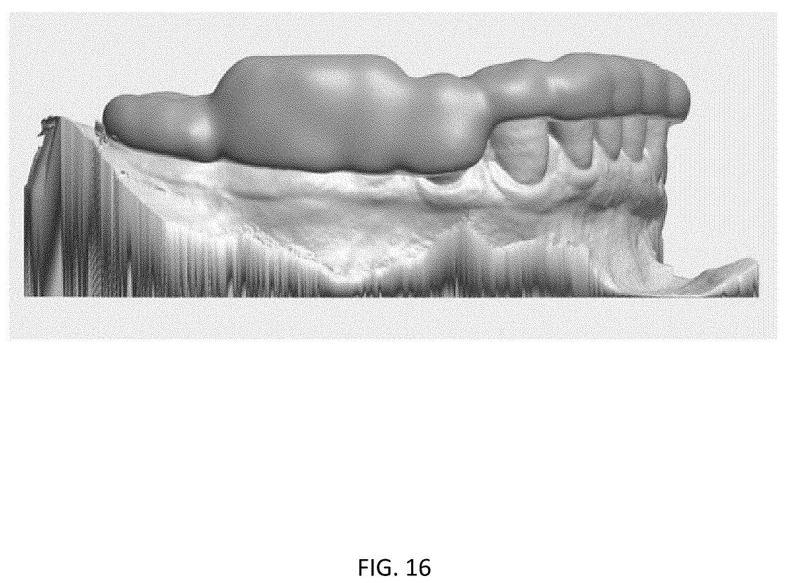

FIG. 16 illustrates an appliance, without the specialized attachments, with posterior bite plane in place according to an example embodiment. A table may be used to show a digital setup of posterior bite plane on the lower or mandibular arch. A computer may be used to generate the table to ensure everything is on the same or even plane. This illustration depicts the side view of the appliance after it has been designed. This digital image of the side view shows a flatness of the posterior table.

FIG. 17 illustrates a specialized attachment placed on an appliance according to an example embodiment. The specialized attachment is placed on a mesh of the appliance in proper orientation, where A and B illustrate distances of approximately 1.0 millimeters from a front body hook to a splint body. This figure shows the specialized attachment being placed in the buccal side wall on the appliance. The specialized attachment must be placed in the correct positioning which is no more than a 1 mm opening, measuring from the ball hook to the side wall. A plane is created on the side wall of the appliance where the specialized attachment is attached and a computer creates digital material support for the attachment, so it does not break off.

Centering the specialized attachment on the upper arch, the attachment may be positioned more gingivally than occlusally with the ball hook facing mesial. The opening between the buccal wall of the glide and the ratatouille ball hook should be no more than 1 mm.

In addition, the lower or mandibular arch the center of the specialized attachment may be needs more occlusally than gingivally positioned with the ball hook facing distally. After fabrication of both arches, elastic bands are added to complete the appliance.

Once the 3D digital arch trays with the specialized attachments are finished printing, the appliance is rinsed in isopropyl alcohol and cured in an ultra-violet light according to manufacturing guidelines.

Once the appliance(s) are fully cured, a technician will remove any supports used in the 3D printing process using flush-cut trimmers. Lab technicians will then adjust the appliance(s) to a desired thickness using a burr on a lathe.

The appliance is then fully sanded with sandpaper using a hand piece. This step is to remove any marks left from the burr and to aid in the polishing of the appliance(s). After it is sanded, the appliance(s) is pumiced using volcanic ash to remove any scratches left from the sandpaper.

Once pumiced and cleaned the appliance gets polished to a high shine using a diamond luster bar. After the finishing is done on the appliance the technician will do a quality check of the appliance on the patients' 3D printed models to check for accuracy and fit of the appliance.

One or more example embodiments described herein may provide an ability for a patient to purchase a kit including a video for easy impression taking; an ability for a user to send an impression to a dental lab for device fabrication, and an ability for a user to receive a manufactured device and elastics for attachment.

One or more example embodiments described herein may provide advantages including, but not limited to: no required specialized training for a dental professional, digital impressions and high-quality digital scans, the ability of a dentist to send a digital scan or impression to an authorized dental lab, the ability of a dental lab to custom fabricate an appliance including upper and lower trays and specialized attachments; the ability of a dental office to fit a patient and add elastic bands, and a patient follow-up in only two weeks.

It may be understood that the example embodiments described herein may be considered in a descriptive sense only and not for purposes of limitation. Descriptions of features or aspects within each example embodiment may be considered as available for other similar features or aspects in other example embodiments.

While example embodiments have been described with reference to the figures, it will be understood by those of ordinary skill in the art that various changes in form and details may be made therein without departing from the spirit and scope as defined by the following claims.

What is claimed is:

1. A dental device comprising:
a unitary upper appliance comprising an upper tray, an upper right specialized attachment disposed on the upper tray and an upper left specialized attachment disposed on the upper tray;
a unitary lower appliance comprising a lower tray, a lower right specialized attachment disposed on the lower tray and a lower left specialized attachment disposed on the lower tray;
at least one elastic attached to the upper right specialized attachment and to the lower right specialized attachment; and
at least one elastic attached to the upper left specialized attachment and to the lower right specialized attachment;
wherein each of the upper right specialized attachment, the upper left specialized attachment, the lower right specialized attachment, and the lower left specialized attachment comprises an open ball hook configured to retain the at least one elastic thereon.
